(19) United States
(12) Patent Application Publication
(10) Pub. No.: US 2002/0065214 A1
(43) Pub. Date: May 30, 2002

(54) Method of treating congestive heart failure

(76) Inventors: Adrian Iaina, Qiriat Ono (IL); Dov Wexler, Tel Aviv (IL); Donald S. Silverberg, Netanya (IL)

Correspondence Address: PILLSBURY WINTHROP, LLP, P.O. BOX 10500, MCLEAN, VA, 22102, US

(21) Appl. No.: 09/725,161

(22) Filed: Nov. 29, 2000

(51) Int. Cl. A61K 38/22; A61K 31/7016; A61K 38/48; A61K 31/704; A61K 31/21

(52) U.S. Cl. 514/2; 514/53; 424/94.63; 514/26; 514/509

(57) ABSTRACT

A method of treating congestive heart failure in a subject suffering therefrom, comprising administering erythropoietin and intravenously administering an intravenously administrable iron compound to the subject. The iron is preferably administered in the form of a complex of a ferric hydroxide with erythropoietin.

The present invention relates to a method of treating congestive heart failure and especially to the improvement of the cardiac function and the functional cardiac class of a subject suffering from congestive heart failure.

Congestive heart failure (CHF) is a major and steadily increasing cause of hospitalization, morbidity and mortality. It results from various heart diseases and its cause and prognosis are influenced by many factors. The clinical manifestations of congestive heart failure are principally related to resultant dysfunctions of various vital organs other than the heart, such as lungs, kidneys and liver. Maeda K, et al. Jpn. Circ. J. 1982; 46: 137-142, describe several different factors (eight variables) contributing to the prognosis of congestive heart failure. One of these variables is the reduced hemoglobin content (Hb) of the patients with congestive heart failure.

Volpe, M. et al., Am. J. Cardiol. 1994; 74: 468-473, describe that the kidney and its related hormonal mechanisms play a fundamental role in the pathophysiology of congestive heart failure. They found that the blood levels of erythropoietin, a hormone produced by the kidney in patients with CHF increased and in certain cases the hemoglobin concentration was reduced.

Anand I. S., et al., Br. Heart J. 1993; 70: 357-362, found that chronic severe anemia is often associated with various degrees of salt and water retention. When fluid retention is severe the condition is referred to as congestive heart failure. Haemodynamic changes are reversed after the anemia is corrected.

Löw I. et al., Clin. Nephrol. 1989; 31: p. 26-30, found that the correction of renal anemia by erythropoietin led to a marked decrease in heart size indicated by a considerable reduction of the left ventricular end-diastolic diameter as well as the end-systolic diameter and the cardiac output decreased. Löw-Friedrich I, et al., Am. J. Nephrol. 1991; 11: p. 57-60 confirmed that the therapy with erythropoietin reduces cardiac size and improves heart function in chronic hemadiolysis patients. Goldberg N., Am. Heart J. 1992; 124: p. 424-427, reported that the enlarged ventricular volumes in chronically anemic patients are reduced by treatment with erythropoietin.

The treatment of iron deficiency and iron deficiency anemia by oral, parenteral or intravenous administration of iron is known. It is also known that renal diseases affect the function of the renal tubules and this impairs the secretion of erythropoietin. The consequence is anemia. It is known that this anemia can be treated by administration of recombinant human erythropoietin in order to replace the missing erythropoietin. It has been recommended to administer iron together with the erythropoietin therapy.

Up to now there have not been any publications describing the treatment of congestive heart failure by application of erythropoietin together with iron. Anemia has been considered to be only a rare contributing factor to the worsening of congestive heart failure and estimated as contributing to no more than 0 to 1.5% of all cases. (Ghali J. K. et al., Arch Intern Med 1988; 148: p. 2013-2016; Opasich C. et al., Am J Cardiol 1996; 78: p. 354-357; Michaelsen A. et al., Heart 1998; 80: p. 437-441). Thus, the U.S. Public Health Service Guidelines of Treatment of Congestive Heart Failure (No. 11 AHRCPR Publication No. 95-0613, 1994), does not suggest the use of erythropoietin and iron preparations for the prevention and treatment of anemia in congestive heart failure.

In accordance with the present invention it has now been found that mild anemia is an important factor in patients with congestive heart failure. In fact, in one study of 142 patients with congestive heart failure (CHF) the mean hemoglobin was 11.9±1.5 g % and the percentage of patients with anemia (Hb less than 12 g %) measured from 9.1% for mild CHF to 79.1% for severe CHF. The degree of anemia paralleled the decrease in cardiac and renal function and may have contributed to the fall in both cardiac and renal function. It has further been found that erythropoietin and iron supplementation are important in treating the congestive heart failure. The inventor published his invention in June 2000, Journal of the American College of Cardiology, 2000; Vol. 35, No. 7, pages 1737-1744, the content of which is incorporated by reference into this specification.

The present invention is directed to a method of treatment of congestive heart failure in a subject suffering therefrom, comprising administering of erythropoietin and intravenously administering of intravenously administrable iron compound to the subject.

In accordance with the present invention the iron compound is administered intravenously. Iron compounds for intravenous substitution are known. Any iron compounds known for this purpose can be used. Specific examples of iron compounds are water soluble iron compounds, especially water soluble complexes of ferric compounds, e.g. ferric hydroxide. Such complexes are for example complexes with hydrocarbons, such as iron (III) hydroxide/dextrane complexes as described in U.S. Pat. No. 4,599,405, which is incorporated by reference into this specification.

In accordance with a preferred embodiment of the present invention, the water soluble iron compounds are complexes of iron (III) hydroxide with sucrose. Preparations containing such complexes are commercially available; they can, e.g. be prepared in accordance with Nissim J. A., THE LANCET, Apr. 23, 1949, p. 686-689, or Müller A., Arzneim.-Forsch. (Drug Res.) 24, No. 6, 1974, p. 880-883. The iron (III) hydroxide complexes with sucrose are especially polynuclear iron complexes similar to ferritin (iron (III)-hydroxide phosphate protein complex), the physiologically occurring iron storage protein, wherein the protein ligand apoferritin is replaced by a carbohydrate component.

Iron sucrose complexes contain iron in a nonionic form. The polynuclear iron (III) hydroxide cores are superficially surrounded by a large number of non-covalently bound sucrose molecules. A commercially available iron sucrose complex is Venofer®, produced and sold by Vifor (International) Inc., Switzerland. This specific preparation contains an iron complex having a large weight average molecular mass (Mw) of approximately 43 kDa which is not secreted through the renal passway (Danielson B. G. et al, Drug Res., 1996; 46: p 615-621). Its formula can be summarized as [(Na_{2.3}[Fe₅O₈(OH)_{1.3}(OH₂)_{2.7}][C₁₂H₂₂O₁₁]_{13.05})+0.9Na OH+0.1 NaCl]ₙ n=43/5.

The erythropoietin can be administered in the form of commercially available preparations, like preparations of recombinant human erythropoietin. It can be administered intravenously or subcutaneously.

The water soluble iron compound as well as the erythropoietin are administered in such a way that the desired effect is achieved in accordance with the state of the subject (human or animal) to be treated. For example, the erythropoietin can be administered in doses of about 500 IU to 10000 IU per week. The iron compound is for example administered in doses of about 100 to 200 mg iron two or three times a week.

The total cumulative dose of iron sucrose (e.g. Venofer) can for example be determined equivalent to the total iron deficit (mg) by the hemoglobin level and body weight. The dose and dosage schedule of iron can, e.g. be calculated using the following formula:

Total iron deficit [mg]=body weight [kg]×(target Hb−actual Hb) [g/l]×0.24* +depot iron [mg]

* Factor 0.0034×0.07×1000 (iron content of hemoglobin≅0.34%; Blood volume≅7% of body weight; Factor 1000 conversion from g to mg)

Up to 35 kg body weight: target Hb=130 g/l and depot iron=15 mg/kg body weight

Above 35 kg body weight: target Hb=150 g/l and depot iron=500 mg

The treatment of the present invention can be applied together with usual treatment of congestive heart failure, e.g. by application of angiotensin-converting enzyme (ACE) inhibitors, alpha-and beta-blockers, long-acting nitrates, digoxin, aldactone and firosemide (oral and i.v.).

### EXAMPLES

### Method

26 congestive heart failure (CHF) patients were treated. All patients received a combination of erythropoietin (EPO) subcutaneous and iron (Fe) intravenous. The EPO was given once a week at a starting dose of 2000 IU per week subcutaneously and the dose was increased or decreased as necessary to achieve and maintain a target Hb of 12 g %. The Fe (Venofer of Vifor (International), St. Gallen, Switzerland), a ferric sucrose complex, was given in a dose of 200 mg IV in 150 ml saline over 60 minutes every week until the serum ferritin reached 400 μg/l or the percent Fe saturation (serum/iron total iron binding capacity×100) reached 40% or until the Hb reached 12 g %. The Fe was then given at longer intervals as needed to maintain these levels.

The study was carried out over a range of 4 to 15 months.

### Results

During the treatment period the New York Heart Association (NYHA) class fell from a mean of 3.66 to 2.66. The mean left ventricular ejection fraction (LVEF) increased from 27.7 to 35.4%. Compared with a similar period of time before the onset of the treatment, the mean number of hospitalizations fell from 2.72 to 0.22 per patient (decrease of 91.9%). No significant changes were found in the mean systolic/diastolic blood pressure. Rather surprisingly it has been found that an improvement in cardiac function occurred even if the baseline anemia was quite modest (mean Hb 10.16 g %) and the improvement in the Hb level was only about 2 g %. In accordance with the present invention an impressive improvement in cardiac function can be achieved which is reflected in a marked improvement of the NYHA functional class and a striking reduction in hospitalizations as well as a reduction of the use of oral and IV furosemide preparations.

1. A method of treating congestive heart failure in a subject suffering therefrom, comprising administering erythropoietin and intravenously administering an iron compound to the subject, the erythropoietin and iron compound being administered in amounts sufficient to improve cardiac function and functional cardiac class of the subject.

2. The method of claim 1, wherein the iron compound is a water soluble complex of a ferric hydroxide.

3. The method of claim 2, wherein the complex of a ferric hydroxide comprises a carbohydrate.

4. The method of claim 3, wherein the carbohydrate is sucrose.

5. The method of claim 1, wherein the erythropoietin is recombinant human erythropoietin.

6. The method of claim 1, wherein the erythropoietin is administered subcutaneously.

7. The method of claim 1 further comprising administration of a least one member selected from the group consisting of angiotensin-converting enzyme (ACE) inhibitors, alpha blockers, beta blockers, long-acting nitrates, digoxin, aldactone and furosemide.
